(19) United States
(12) Patent Application Publication
(10) Pub. No.: US 2010/0076298 A1
(43) Pub. Date: Mar. 25, 2010

(54) METHOD AND DEVICE FOR DETERMINING THE SPATIAL DISTRIBUTION OF THE SPECIFIC ABSORPTION RATE PRODUCED BY AN ELECTROMAGNETIC FIELD-RADIATING APPARATUS

(76) Inventor: PATRICK GROSS, Langensendelbach (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/561,442

(22) Filed: Sep. 17, 2009

(30) Foreign Application Priority Data
Sep. 17, 2008 (DE) 10 2008 047 646.3

(51) Int. Cl. A61B 5/055

(52) U.S. Cl. 600/411

(57) ABSTRACT

In a method to determine the spatial distribution of the specific absorption rate in tissue that represents a measure of the absorption of electromagnetic fields emitted by means of a radiation generating element, at least one item of measurement information acquired by a thermoacoustic computed tomography device is used to determine the specific absorption rate.

### BACKGROUND OF THE INVENTION

1. Field of the Invention

The invention concerns a method to determine the spatial distribution of the specific absorption rate in tissue that represents a measure of the absorption of electromagnetic fields emitted by means of a radiation generating element of a medical device.

2. Description of the Prior Art

The signal-to-noise ratio (also called SNR) of the magnetic resonance signal that is detected in a magnetic resonance apparatus represents a basic problem in magnetic resonance measurements. In order to increase the SNR of a magnetic resonance measurement, one possibility is to increase the magnetic field strength B0 of the basic field magnet. However, the duration of an RF pulse that must be radiated in order to achieve a specific flip angle of the magnetization is also extended at higher field strengths, or the power of this radiated RF pulse must be increased. This means that more energy must be introduced into the examination subject at higher field strengths, given otherwise identical image acquisition parameters, in order to implement measurements by means of a specific measurement sequence. This problem also depends on the pulses that are used, which is why spin echo-based sequences pose particular problems due to the use of refocusing pulses therein.

In addition to the RF energy introduced into the examination subject, with increasing field strengths the dielectric properties of the tissue coming into contact with the RF energy are no longer negligible. Therefore it is impossible to deduce the heating of the tissue as a whole from the RF energy introduced into the tissue via the radio-frequency coil. Rather, individual points in the tissue are heated much more significantly than others due to their individual dielectric properties and conductivity.

The specific absorption rate is defined as a measure of the absorption of electromagnetic fields in biological tissue. As a result of the interaction of the electromagnetic fields with the tissue, the tissue heats depending on its dielectric properties. A precise knowledge of the specific absorption rate (also abbreviated as SAR) thus allows the calculation of the permissible interactions between electromagnetic fields and tissue (and thus a prediction of the heating of the examination subject). For this reason, local and whole body SAR are used as regulatory values, for example by the IEC (International Electrotechnical Commission). A precise determination of the specific absorption rate is not possible according to the prior art, which is why workaround solutions are applied.

To avoid this problem, it is known to furthermore globally calculate the specific absorption rate of the tissue and to decrease its limit value so far that even the possible occurrence of heating spikes in the tissue cause no damage. A monitoring of the heating of the examination subject by means of MR temperature imaging is also problematic since only heating in the range of multiple degrees Celsius can be significantly detected, but local temperature spikes can be averaged out by partial volume effects.

It is also known to use pulse sequences that require a lower RF energy for implementation given the same information content. The case of the spin echo sequences is cited again as an example. For turbo-spin echo sequences it is known to use pulses with smaller flip angles for refocusing instead of the 180° refocusing pulses that are problematic from an SAR standpoint, with the desired contrast being retained through appropriate selection of the flip angle sequence.

### SUMMARY OF THE INVENTION

An object of the invention is to provide a method that allows the determination of the specific absorption rate, in particular even for magnetic resonance measurement at field strengths greater than 1.5 T, for instance.

This object is achieved in a method of the aforementioned type wherein, according to the invention, at least one item of measurement information acquired by a thermoacoustic computed tomography device is used to determine the specific absorption rate.

Thermoacoustic computed tomography is an imaging method in which an examination subject is heated by means of a short heating pulse and the heating tissue expands, causing one or more sound waves to be emitted. These sound waves can then be detected by suitable devices. An image can then be calculated from such data, with the reconstruction proceeding analogously to known methods of x-ray computed tomography; see Kruger et al., “Thermoacoustic CT: Imaging Principles,” Proc. SPIE Vol. 3916, P. 150-159, 2000. The data or the image calculated from the data provide information about the conductivity of the tissue.

Instead of visualizing a spatial distribution of the sound wave pressure, according to the invention the thermoacoustic computed tomography device is used to determine the specific absorption rate.

The spatial distribution of the coefficient of thermal expansion that is detected by the thermoacoustic computed tomography device can be particularly advantageously used as the aforementioned item of measurement information. The coefficient of thermal expansion i.e., its spatial distribution, can be determined by means of multiple post-processing steps from the measurement data acquired by the thermoacoustic computed tomography device. The correlation between the coefficient of thermal expansion and the spatial distribution of the heating of the sample is (Equation 1):

$${p}_{r} ( t ) = \frac{βρ}{r π} \int \int \int \frac{{\partial }^{2} T ( {r}^{′} , {t}^{′} )}{\partial {t}^{2}} \frac{d {r}^{′}}{\vert r - {r}^{′} \vert}$$

wherein pᵣ(t) designates the coefficient of thermal expansion, β is an expansion factor, ρ is the density of the tissue, and T(r′, t′) is the temperature at the location r′.

At least one expansion factor can advantageously be taken into account as additional information. While an image can easily be back-calculated from the measurement data of the thermoacoustic computed tomography device, further information is required for exact quantification of the specific absorption rate. One possibility to arrive at the specific absorption rate from the measurement information is to take into account the expansion factors of the different tissue located in the image acquisition region (in the heating region) according to Equation 1 in order to thus determine the energy absorption of the tissue. For example, the at least one expansion factor of the tissue can be detected by another imaging device. This other imaging device thus must deliver supplementary information with which the expansion factors can be determined.

At least one parameter map acquired with a magnetic resonance device and/or at least one image acquired with a magnetic resonance device can particularly advantageously be used as possible additional further information. A parameter map, what is thereby to mean a series of image data sets that allows the quantification of a specific parameter. For example, this can be the T₁ relaxation time, the T₂ relaxation time, the coefficient of diffusion, the coefficient of perfusion and also any additional parameter known from magnetic resonance tomography. Furthermore, it is possible that the image data may contain additional spectroscopic information, so a quantification of water and fat is possible, for example. Such information can be obtained by means of chemical shift imaging, for example. Moreover, in part it is not always necessary to acquire whole image series for quantification of parameters since a certain parameter weighting can already be achieved in a single image data set. For example, by selection of a sufficiently long echo time, a T₂ weighting can be achieved in a spin echo data set and a T₂* weighting can be achieved in a FLASH data set. Conclusions of tissue distributions thus can already be achieved from the single data set. The spatial distribution of the magnetic field B₁⁺ generated by the radio-frequency coil used for magnetic resonance measurement can be taken into account as possible additional further information. The magnetic resonance device is thus used to determine the applied RF field. Correction information resulting from this can be used to further improve the quantification of the specific absorption rate.

Information about the spatial distribution of the dispersion and/or the speed of sound and/or the absorption of the tissue can be determined to further improve the determination of the specific absorption rate. The more information that exists with regard to the parameters affecting the sound propagation, the more precisely that the determination of the specific absorption rate can be implemented.

The heat pulse necessary to implement the thermoacoustic computed tomography can be particularly advantageously applied with a radio-frequency coil suitable for magnetic resonance imaging. In order to be able to ignore diffusion effects, the duration of such a heat pulse is in the range of one microsecond, for example 500 nanoseconds. Although typical RF pulses that are used for magnetic resonance imaging are in the range of one millisecond, the radio-frequency coils can likewise generate shorter pulses. Since the performance and efficiency of a number of coil models are known due to decades of research, in practice known coils and pulse designs can thus be used. The heat pulse can advantageously be applied at essentially the same frequency as the resonance frequency used for magnetic resonance measurement. This offers the advantage that the same frequency spectrum as in magnetic resonance measurement is used to determine the specific absorption rate. Thus, with regard to the transferability of the specific absorption rate determined via the thermoacoustic computed tomography to the effect of the RF pulses emitted by the radio-frequency coil, no frequency-dependent corrections are necessary.

Alternatively, the heat pulse can be applied with a resonance frequency that is different from the resonance frequency used for proton magnetic resonance measurement, but that corresponds to a different atomic nucleus. This procedure offers the advantage that the measurements can be implemented simultaneously and independently of one another by means of magnetic resonance tomography and thermoacoustic computed tomography. Given this frequency choice, dual-resonance coils that may be present can be used, wherein one resonance frequency is used for magnetic resonance imaging and the second resonance frequency is used for thermoacoustic computed tomography. Using the repetition time of the magnetic resonance measurement, sufficient wait periods are typically available in which measurements at another resonance frequency can then occur without affecting the results of the magnetic resonance measurement at its natural frequency.

The above object also is achieved in accordance with the present invention by a combined medical device that includes the components described above, and that operates in accordance with the method described above, as well as all embodiments of that method.

### BRIEF DESCRIPTION OF THE DRAWINGS

FIG. 1 shows a basic representation of a combined medical device according to the invention.

FIG. 2 is a cross-section through a combined detection unit in the medical device according to the invention.

FIG. 3 shows basic steps in an embodiment of the method according to the invention.

FIG. 4 schematically illustrates the use of the inventive method in the environment of a radar apparatus.

FIG. 5 schematically illustrates the use of the inventive method in the environment of a mobile telephone.

### DESCRIPTION OF THE PREFERRED EMBODIMENTS

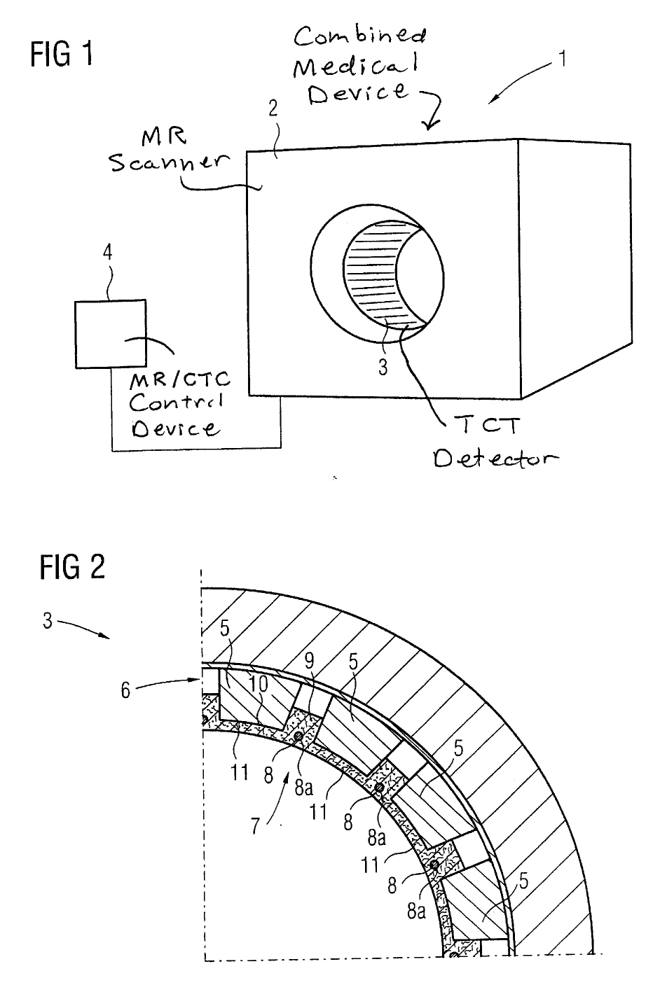

The magnetic resonance (MR) scanner 2 of the combined medical device 1 shown in FIG. 1 also has components of a thermoacoustic computed tomography (TCT) device integrated therein. For data acquisition, a combined MR/TCT detection unit 3 is provided while the data are evaluated by a common MR/TCT control device 4. Further known components of a magnetic resonance system—for instance in the form of gradient coils, preamplifiers, a cooling device etc. —are not individually shown since they are well known to those skilled in the art.

As shown in FIG. 2, the combined MR/TCT detection unit 3 units the transmission and reception unit of the magnetic resonance system 2 with a detection system 6 of the thermoacoustic computed tomography device. The detection system 6 of the thermoacoustic computed tomography device includes an annular detector arrangement with detector blocks 5 as well as a radio-frequency coil arrangement 7 with longitudinal conductors 8. This radio-frequency coil arrangement 7 is arranged within the TCT detector system 6 and is coaxial therewith. The longitudinal conductors 8 are separated by the boundary surfaces of the interstices 9. The longitudinal conductors 8 are advantageously directed within the interstices 9 at least over a portion of their radial cross section. A central arrangement with regard to the interstices is desirable.

At least the segments 8a of the longitudinal conductors 8 directed along the interstices 9 can be fashioned as wires with a round cross section. The spacing of the wires from a radio-frequency shield 10 shielding the radio-frequency coil arrangement 7 from the TCT detector system 6 should amount to at least the value of their own diameter, but advantageously to 5 to 10 times their own diameter. The shown detector arrangement in annular form is only an example; other designs are possible without further measures.

A suitable coil type is the birdcage resonator. Such resonators possess ferrules at the end of the longitudinal conductor 8 that are then to be arranged before and after the TCT detector system 6 in an axial direction. These ferrules and the segments of the longitudinal conductors 8 not running within the TCT detector system 6 can be executed as flat conductors, in particular as copper foils. The interstices 9 are filled with a dielectric material 11, whereby the longitudinal conductors 8 can also be mounted.

An extremely compact design of the combined medical device 1 is possible with such a combined detection unit 3. In this embodiment, a registration of the respective acquired measurement data is also then possible without problems, and the control by means of a single control device 4 then also does not represent a problem.

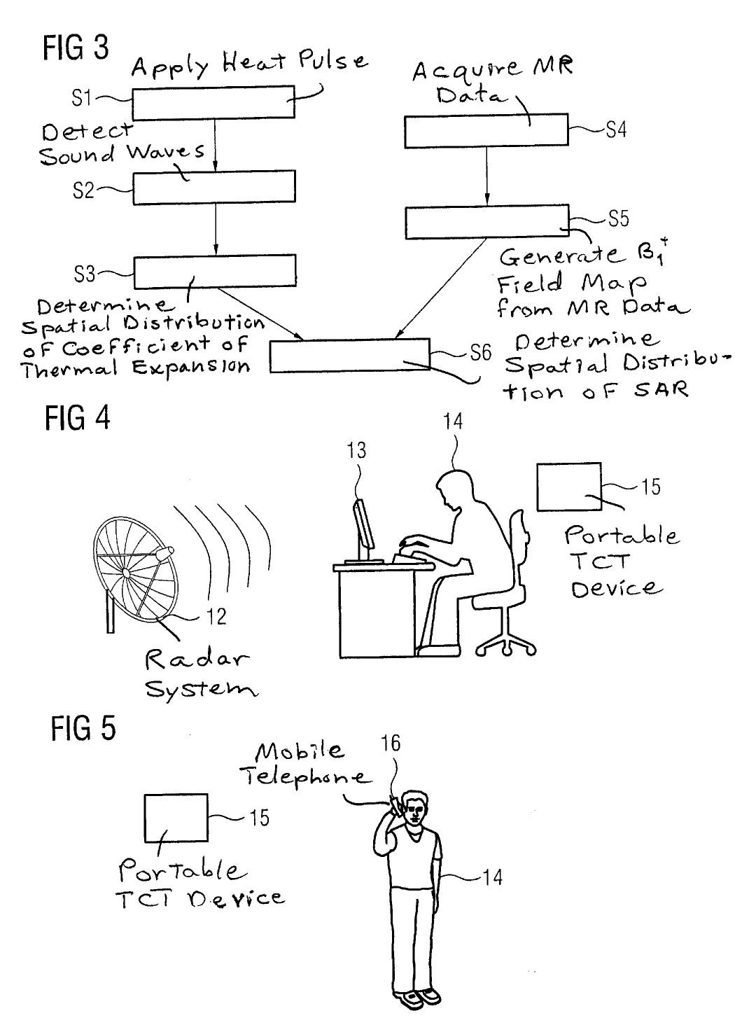

The basic step 6 to determine the specific absorption rate are shown in FIG. 3. In Step S1 a heat pulse is introduced into the examination subject by means of the radio-frequency coil arrangement 7, and the sound waves that are thereby generated are acquired by the TCT detector system 6 in Step 2. In Step S3, the spatial distribution of the coefficient of thermal expansion is determined from these sound waves. Depending on the design of the TCT detector system 6, this information can be two-dimensional or three-dimensional.

Following this or simultaneously, in step S4 a data set composed of multiple images is acquired by means of the magnetic resonance system to determine the spatial distribution of the B₁⁺ magnetic field. Not only is a map of the B₁⁺ magnetic field created from this data set in Step S5, but also the expansion factor is determined per pixel as additional information. The spatial distribution of the specific absorption rate of the examination subject can then be obtained from all of this information in a concluding Step S6.

The pulse duration, the pulse damping or even the repetition time or the number of slices to be measured can then be selectively set with the aid of the knowledge about the spatial distribution of the SAR as well as of the B₁⁺ magnetic field such that no harmful heating of the examined tissue is created by means of the RF energy induced by the radio-frequency coils. Thus instead of calculating a global SAR and establishing the limit values correspondingly low, more precise information is provided for setting the SAR limit value. As a result, an automated SAR monitoring can be achieved to improve the patient protection while expanding the possibilities for examination.

Additional usage scenarios of the method are also conceivable. For example, the SAR can also be measured at a radar system 12, as shown in FIG. 4. If it is desired to make such a measurement for a person 14 at a workstation 13 of a radar system 12, a portable TCT device 15 is required. An SAR determination can be implemented with this at arbitrary usage locations.

The method according to the invention is also suitable for SAR determination in the use of a mobile telephone 16 (see FIG. 5). Here as well as a portable TCT device 15 is used.

The method is not limited to these fields of use. A problem with regard to the energy absorption of tissue is also known given use of WLAN, Bluetooth, radio waves or hyperthermia systems, so an SAR determination in the manner according to the invention can also be beneficially used in such sceneries.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method to determine the spatial distribution of the specific absorption rate (SAR) in biological tissue, comprising the steps of:
obtaining measurement information from a subject containing biological tissue by thermoacoustic computed tomography (TCT); and
in a processor, automatically determining a spatial distribution of the SAR in the subject using said measurement information obtained by TCT.

2. A method as claimed in claim 1 comprising, in said processor, automatically determining said spatial distribution as a distribution of the coefficient of thermal expansion represented by said measurement information obtained by TCT.

3. A method as claimed in claim 1 comprising, in said processor, determining said spatial distribution of the SAR by identifying an expansion factor associated with said biological tissue and additionally using said expansion factor to determine the spatial distribution of the SAR.

4. A method as claimed in claim 3 comprising detecting said expansion factor using an imaging device other than a TCT device.

5. A method as claimed in claim 1 comprising, with a magnetic resonance apparatus, obtaining additional information selected from the group consisting of a parameter map and a magnetic resonance image and, in said processor, using said additional information together with said measurement information obtained by TCT to automatically determine the spatial distribution of the SAR.

6. A method as claimed in claim 5 comprising, in said magnetic resonance apparatus, employing a radio-frequency coil that generates a B⁺₁ magnetic field having spatial information associated therewith, and using said spatial information associated with said B⁺₁ magnetic field as said additional information.

7. A method as claimed in claim 4 comprising, with said imaging device, providing information, as said additional information, selected from the group consisting of a spatial distribution of dispersion, a speed of sound, and absorption of tissue in the subject.

8. A method as claimed in claim 1 comprising placing said subject in an MR imaging apparatus having a radio-frequency coil used by said imaging apparatus to acquire said MR measurement data, and administering a heat pulse to the subject in said TCT using said radio-frequency coil of said MR imaging apparatus.

9. A method as claimed in claim 8 comprising operating said radio-frequency coil of said MR apparatus at a frequency for acquiring said MR measurement data, and applying said heat pulse at substantially the same frequency in said TCT.

10. A method as claimed in claim 8 comprising operating said radio-frequency coil of said MR apparatus at a first frequency to excite hydrogen nuclei for obtaining said MR measurement data, and operating said radio-frequency coil at a second frequency, for a different nucleus, to generate said heat pulse in said TCT.

11. A medical device comprising:
a magnetic resonance (MR) scanner, configured to receive an examination subject therein containing biological tissue, said MR scanner being configured to acquire MR measurement data from the examination subject;
a thermoacoustic computed tomography (TCT) detector system integrated into said MR scanner, that acquires TCT measurement data from the examination subject in the MR scanner; and
a computerized evaluation unit, supplied with said MR measurement data and said TCT measurement data, which automatically determines a spatial distribution of the specific absorption rate of the biological tissue in the examination subject using said TCT measurement data.

12. A medical device as claimed in claim 11 wherein said MR scanner comprises a radio-frequency coil used by said MR scanner to acquire said MR measurement data operable to and administer a heat pulse to the subject in acquiring said TCT measurement data.

13. A medical device as claimed in claim 12 wherein said MR scanner operates said radio-frequency coil at a frequency for acquiring magnetic resonance data, and applies said heat pulse at substantially the same frequency to acquire said TCT measurement data.

14. A medical device as claimed in claim 12 wherein said MR scanner operates said radio-frequency coil at a first frequency to excite hydrogen nuclei for obtaining said MR measurement data, and operates said radio-frequency coil at a second frequency, for a different nucleus, to generate said heat pulse to acquire said TCT measurement data.

15. A medical device as claimed in claim 11 wherein said computerized evaluation unit determines said spatial distribution of the specific absorption rate by mapping said TCT measurement data with said MR measurement data.
